(19) United States
(12) Patent Application Publication
(10) Pub. No.: US 2014/0228609 A1
(43) Pub. Date: Aug. 14, 2014

(54) METHOD AND SYSTEM FOR PRODUCING OLEFINS FROM DIMETHYL ETHER

(71) Applicants: Gerhard Birke, Frankfurt (DE); Bernd Ahlers, Dietzenbach (DE); Harald Kömpel, Neu-Isenburg (DE); Waldemar Liebner, Oberursel (DE)

(72) Inventors: Gerhard Birke, Frankfurt (DE); Bernd Ahlers, Dietzenbach (DE); Harald Kömpel, Neu-Isenburg (DE); Waldemar Liebner, Oberursel (DE)

(21) Appl. No.: 14/347,066

(22) PCT Filed: Aug. 16, 2012
(86) PCT No.: PCT/EP2012/066022

§ 371 Date: Mar. 25, 2014

(30) Foreign Application Priority Data
Sep. 27, 2011 (DE) 10 2011 114 367.3

(51) Int. Cl. C07C 1/22

(52) U.S. Cl. CPC C07C 1/22
USPC 585/639; 422/111

(57) ABSTRACT

For the production of olefins from dimethyl ether gaseous dimethyl ether in a purity of 70-100 wt-% together with recycle gas, which contains olefinic, paraffinic and/or aromatic hydrocarbons, as well as steam is charged to a first catalyst stage of a re actor.

To render the temperature profile over the catalyst stages as flat as possible, but close to the optimum operating temperature, gaseous dimethyl ether in a purity of 70-100 wt-% together with recycle gas, which contains olefinic, paraffinic and aromatic hydrocarbons, is charged to at least one downstream catalyst stage, wherein this downstream catalyst stage additionally is fed with product gas from the upstream catalyst stage.

### CROSS-REFERENCE TO RELATED APPLICATIONS

This application is a national stage application under 35 U.S.C. 371 of International Patent Application Serial No. PCT/EP2012/066022, entitled “VERFAHREN UND ANLAGE ZUR HERSTELLUNG VON OLEFINEN AUS DIMETHYLETHER” filed Aug. 16, 2012, which claims priority from German Patent Application No. DE 10 2011 114 367.3, filed Sep. 27, 2011, the disclosures of which are hereby incorporated by reference herein in their entirety.

### FIELD OF THE INVENTION

The invention relates to the production of olefins from dimethyl ether.

### BACKGROUND

Short-chain olefins, in particular propylene (propene), belong to the most important basic substances of the chemical industry. Proceeding from these unsaturated compounds with short chain length, molecules with longer-chain carbon skeleton and additional functionalizations can be constructed.

As a source for short-chain olefins, the process of steam cracking, i.e. the thermal decomposition when processing petroleum, has been used above all in the past. In recent years, however, further processes for the production of short-chain olefins have been developed. On the one hand, this is due to the rising demand which no longer can be covered by the existing sources; on the other hand, the increasing shortage of fossil raw materials requires the use of other starting substances.

The so-called MTP (Methanol-to-Propylene) or also MTO (Methanol-to-Olefin) processes for producing propylene and other short-chain olefins proceed from methanol as starting substance. In these heterogeneously catalyzed processes, the intermediate product dimethyl ether (DME) initially is partly formed from methanol, and from a mixture of methanol and dimethyl ether a mixture of ethylene and propylene then is formed.

In the classical MTP process, pure methanol is starting substance for the conversion; the same initially is obtained from crude methanol by distillation. Then, methanol is evaporated under a high energy expenditure and in just one cycle is guided to the DME reactor in which it is converted to dimethyl ether and water. The product mixture thus obtained contains methanol/DME in a ratio of about 30:70 wt-% and is cooled and partly condensed.

Together with gaseous dimethyl ether/methanol, the water-rich condensate from the product of the DME reactor is charged to the individual stages of the reactor, where methanol and dimethyl ether are converted to chiefly olefinic hydrocarbons. The product mixture obtained in this conversion subsequently must be subjected to a further purification.

DE 11 2006 002 599 T5 for example describes how above all ethylene can be obtained by corresponding process parameters and can efficiently be purified by connecting rectification means.

A cause for the formation of a product mixture above all is a distinctly pronounced heat profile over the individual catalyst stages in the olefin reactor. Attempts are made to counteract this by charging the educts partly in liquid form. As a result of the evaporation of condensate, the gas stream is cooled before entry into the next catalyst bed. The reaction control nevertheless remains difficult due to the exothermicity of the reactions taking place in the reactor, in particular of the reactions proceeding from methanol. This results in a strong heat tonality inside the individual catalyst stages and in the related disadvantages, and in particular leads to catalyst damages and a decrease in selectivity based on the desired product.

From DE 695 199 99 T2 it is known that into a reactor for producing olefins a mixture of alkoxy compounds, such as e.g. methanol and dimethyl ether, can be introduced, wherein a dilution gas is admixed to this mixture. By the dilution gas it can be achieved that the temperature of the added gas stream lies between roughly 340 and 400° C. and the specific thermal capacity of the resulting gas stream is changed such that the temperature increase inside the gas stream is less than 100° C. It thereby is avoided that local heat zones are formed, which damage the catalyst and in which undesired reactions take place, but at the same time this temperature lies distinctly below the optimum operating temperature. To achieve a comparable conversion, the residence time must be increased. Hence, the conversion per unit time is decreased and accordingly the process efficiency.

The methanol contained in the intermediate DME product in the classical MTP process in so far has a disadvantageous effect as the conversion in the olefin reactor is effected by a strongly exothermal reaction. When using a methanol-DME mixture, the heat tonality of the reactor is distinctly higher than when using pure dimethyl ether. With a conventional execution of the MTP process, switching to pure dimethyl ether, however, is expensive and not economic, because two separate distillation systems for the separation of crude methanol/pure methanol and of DME/methanol/H₂O must be carried out with high investment effort and consumption of auxiliaries.

In a process described in DE 10 2008 058 931 B4, the purification of dimethyl ether advantageously is linked with the purification of the crude methanol. In a reactor, a mixture of methanol, dimethyl ether and water is produced by a heterogeneously catalyzed reaction, which mixture must be processed with regard to the separation of the individual constituents. For this purpose, the mixture from the reactor is supplied to a first distillation column in which a mixture of methanol and dimethyl ether is separated from the bottom product substantially consisting of water. The methanol-DME mixture is supplied to a second distillation column in which the purified dimethyl ether is withdrawn over head. The bottom product consisting of methanol for the most part is supplied to a third distillation column, which at the same time is fed with the starting substance crude methanol. The methanol purified in this third column can be used as educt for the reactor for the conversion of dimethyl ether. By this method, the methanol distillation and the purification of the dimethyl ether thus can be combined in one process.

The process known from DE 10 2008 058 931 B4 allows to use dimethyl ether as the only feed component for the conversion to olefins and at the same time ensure the economy of the process.

### SUMMARY

Proceeding therefrom, it is the object underlying the present invention to convert purified dimethyl ether in several catalyst stages to olefins in a heterogeneously catalyzed process, wherein the temperature profile over the catalyst stages should be as flat as possible, but close to the optimum operating temperature.

According to the invention, the gaseous dimethyl ether with a purity of 70 wt-%-100 wt-%, such as 85 wt-%-99 wt-%, furthermore such as with a purity of at least 95 wt-%, such as of at least 97.5 wt-% is charged to a catalyst stage in a mixture with recycle gas. This provides for an optimized reaction control, in particular it is no longer necessary to introduce a water/methanol mixture in liquid form upstream of the individual catalyst stages and utilize the cooling effect of the evaporation.

Furthermore, it was found to be advantageous to connect several catalyst stages in series inside a reactor and connect the same with each other such that product gas is fed from one catalyst stage into another catalyst stage. This has the advantage that unused educt from the product mixture of the upstream catalyst stage can be converted in the downstream catalyst stage, whereby the yield of the process can further be increased.

To the first catalyst stage, recycle gas and steam are charged. The recycle gas can contain olefinic as well as paraffinic and/or aromatic hydrocarbons.

The steam which is added to the recycle gas before heating to reaction temperature serves as heat carrier and moderator and reduces coking at high temperatures and hence also a premature deactivation of the catalyst. The addition of steam either can be effected directly into the catalyst stage or as mixed stream with the recycle gas. The addition of a single stream facilitates the procedure and proceeds particularly favorably when the recycle gas is heated to the reactor inlet temperature in an undergrate firing unit by adding up to 60 wt-% of steam based on the recycle gas.

To be able to adjust rather homogeneous conditions in each catalyst stage, it is also recommendable to mix this stream of recycle gas and steam with rather gently preheated dimethyl ether and thus charge only one entire stream to the first catalyst stage. The amount of dimethyl ether in relation to the recycle gas at the entrance to the first catalyst stage can be in the range from 5-20 wt-%, particularly at 10 to 15 wt-%.

It was found to be favorable to adjust the ratio of the gas streams charged to the first catalyst stage such that the amount of dimethyl ether in relation to the recycle gas on entry into the catalyst stage is 2 to 20 wt-% or 5-15 wt-%, and the amount of steam in the entire stream on entry into the catalyst stage maximally is 50 wt-%.

To the second and each further catalyst stage, the product gas of the preceding stage is charged and a mixture of dimethyl ether and recycle gas in a ratio of 10-200 wt-%, such as 15-50 wt-% of recycle gas to dimethyl ether. Here as well, the individual streams can be charged separately in principle.

By (finely) adjusting the ratio of dimethyl ether and recycle gas to each other as well as the inlet temperatures of one or both streams, it can be achieved that the inlet temperature into a catalyst stage lies between 420 and 500° C., such as between 440 and 490° C., and/or the outlet temperature from the catalyst stage lies between 450 and 530° C., such as between 460 and 520° C. At these temperatures an almost complete conversion of the dimethyl ether occurs, without this leading to a reduced selectivity with regard to the desired products, above all ethylene and propylene. These temperatures distinctly lie above those which are practiced in conventional processes (inlet temperature about 400° C.).

The inlet temperature is obtained as mixing temperature of the dimethyl ether with the recycle gas and the product stream from an upstream catalyst stage. The outlet temperature results from the inlet temperature into a catalyst stage as well as the temperature increase as a result of exothermal reactions, wherein this exothermicity partly can be compensated by endothermicity of other reactions.

In addition, it is advantageous when the ratio of dimethyl ether and recycle gas is regulated or controlled such that the temperature inside a catalyst bed rises by at least 5° C., but maximally 100° C., such as 40° C. This has the advantage that the formation of so-called hot spots, i.e. locally heated points inside the catalyst stage, can reliably be avoided. The useful life of the catalyst thereby is improved, since the same does not age prematurely due to local heating.

In particular, it also is favorable to separately control the charging of each individual catalyst stage with dimethyl ether and recycle gas, as in this way conditions can be adjusted inside each catalyst stage, which with regard to the introduced product gas stream from an upstream catalyst stage lead to a yield maximization.

Advantageously, it thus is also possible to use recycle gas with different compositions, whereby further possibilities for process optimization inside the individual catalyst stages are given. For example, the recycle gas also can contain methanol and the heat tonality connected with the methanol reaction can be introduced in a catalyst stage where this is desired or accompanied by the least disadvantage, e.g. in the first catalyst stage.

According to an aspect of the invention, recycle gas with a different composition is charged to the first catalyst stage, while all succeeding catalyst stages are charged with recycle gas of the same composition. It also is favorable to use recycle gas with a particularly high olefin content, in particular with chain lengths C₄ to C₈. Since these olefins react in the reactor in an endothermal reaction to form propylene, they also serve for cooling the system and for increasing the yield of target product.

In general, dosing into the individual catalyst stages should be effected such that by means of the temperature and reaction control the total yield of propylene is maximized over all catalyst stages. The process efficiency can further be increased in that dosing into the individual catalyst stages is effected such as is required by the aging condition of the respective catalyst. This means in particular that in the case of a catalyst less active as a result of aging processes a higher inlet temperature can be employed. This can be achieved for example by increasing the methanol content in the inlet stream.

The process in particular can be operated with high yield by using a pressure at the inlet of the reactor of 0.8-5.0 bar(a), such as 1.5-3.0 bar(a).

In addition, the process in particular can be operated with good catalyst lifetimes and a high yield of short-chain olefins such as propylene, when a form-selective zeolite material, such as an alumosilicate of the pentasil type ZSM-5 is used as catalyst. The economy of the process moreover can be increased in that the recycle gas is obtained during the purification to which the olefins obtained from dimethyl ether are subjected. Thus, waste waters otherwise obtained in the process can be minimized, or hydrocarbon fractions contained therein partly can yet be converted to a valuable product by again passing through the catalyst stage. Streams with a content of 10-70 wt-%, such as 20-40 wt-% of C₂ and/or C₄-C₈ olefins can be used as recycle gas.

In particular, a gas stream suitable as recycle gas is obtained, when in such processing unit the hydrocarbons produced are purified with methanol as washing agent. The methanol used after its use as washing agent can be processed in a distillation unit. The top product of this distillation unit then can at least partly be guided directly to the reactor as recycle gas, such as recycle gas to at least one of the catalyst stages downstream of the first catalyst stage.

Advantageously, the methanol which is produced as bottom product of this distillation column also can be recycled, in that it is supplied to the DME reactor. This is accomplished in that this bottom product is recirculated as reflux into that distillation which also serves for purifying the crude methanol for the conversion to DME.

Furthermore, it was found to be favorable to carry out a method for producing pure dimethyl ether from crude methanol before the method according to the invention. For this purpose, the crude methanol produced in a first process step is supplied to an at least one-stage distillation stage and from there, in a second process step, converted to dimethyl ether inside a reaction stage. Subsequently, the dimethyl ether thus obtained is purified in an at least one-stage, such as at least two-stage distillation, wherein recovered methanol is recirculated into that distillation which also serves for purifying the crude methanol.

In an embodiment, the two-stage distillation is designed such that within a first purification step water is separated from a methanol/dimethyl ether mixture and in a second distillation the methanol/dimethyl ether mixture subsequently is separated with respect to the two main components.

The invention furthermore comprises a plant for the production of olefins from dimethyl ether, which is suitable for carrying out the process according to the invention. Such plant includes a reactor with at least two catalyst stages. At least one supply conduit for dimethyl ether, recycle gas and steam opens into at least one catalyst stage, wherein these components can be introduced as mixture or individually. Into at least one downstream catalyst stage connected with the first stage, recycle gas is introduced via a conduit. In addition, via this one or a second conduit dimethyl ether is supplied. The recycle gas each used contains olefinic, paraffinic and aromatic hydrocarbons. For dosing the recycle gas in relation to the used dimethyl ether, at least one control or regulating device is provided in each supply conduit.

In an embodiment, the control device is designed such that the temperatures of the inlet stream into the respective catalyst stage and of the corresponding outlet stream are measured and used as control quantities for the amounts of recycle gas and dimethyl ether to be dosed, as in this way the parameter decisive for the reaction control, the temperature, can directly be used as control quantity. In accordance with a development of the invention, temperature measuring points also can be provided inside the catalyst bed of the respective catalyst stage, and the temperatures determined there can be used as control quantity.

According to the invention, it was found to be favorable with such interconnection of several successive catalyst stages, when each catalyst stage is equipped with its own supply conduit for recycle gas and at least one associated control or regulating means, as in this way the process conditions of the individual catalyst stages can be controlled independently.

An advantageous embodiment of the invention in addition provides that from at least one means for the treatment of the olefins a conduit leads to the supply conduit for the recycle gas.

When the purification of crude methanol should be linked with the purification of the dimethyl ether, the plant according to the invention includes a distillation means for the purification of crude methanol and a reactor for the conversion of crude methanol to dimethyl ether. Furthermore, such plant contains at least two distillation means for purifying the dimethyl ether, wherein one of the further distillation means is linked with the reactor for the olefin production via a conduit. In addition, a conduit leads from one of the further distillation means back to the first distillation means for the purification of crude methanol.

In an embodiment, the invention provides for a process for producing olefins from dimethyl ether, wherein gaseous dimethyl ether in a purity of 70-100 wt-%, such as 85-99 wt-% together with recycle gas which contains olefinic, paraffinic and/or aromatic hydrocarbons, as well as steam is charged to a first catalyst stage of a reactor and wherein gaseous dimethyl ether in a purity of 70-100 wt-%, such as 85-99 wt-%, together with recycle gas which contains olefinic, paraffinic and aromatic hydrocarbons is charged to at least one downstream catalyst stage, wherein this downstream catalyst stage additionally is fed with product gas from the upstream catalyst stage.

In an embodiment, the ratio of the gas streams charged to the first catalyst stage is adjusted such that the amount of dimethyl ether in relation to the recycle gas on entry into the catalyst stage is about 2 to 20 wt-%, such as 5-15 wt-%, and the amount of steam in the entire stream on entry into the catalyst stage maximally is 60, such as 50 wt-%, and/or that the ratio of the gas streams charged to a downstream catalyst stage is adjusted such that the amount of the recycle gas in relation to the dimethyl ether on entry into the catalyst stage is 10-200 wt-%, such as 15-50 wt-%.

In an embodiment, the ratio of dimethyl ether and recycle gas is adjusted such that the inlet temperature into the catalyst stage lies between 440 and 490° C. and/or the outlet temperature from the catalyst stage lies between 460 and 520° C.

In an embodiment, the ratio of dimethyl ether and recycle gas is adjusted such that the temperature over the catalyst stage rises by 5 to 100° C., such as 5 to 40° C. or 10-25° C.

In an embodiment, recycle gas with a different composition is charged to the first catalyst stage, while all succeeding catalyst stages are charged with recycle gas of the same composition.

In an embodiment, a mixture of dimethyl ether and recycle gas is charged to at least two catalyst stages, wherein the respectively added recycle gas has a different composition.

In an embodiment, for the last catalyst stage the ratio of dimethyl ether and recycle gas is adjusted such that the temperature over the catalyst stage rises by 5 to 20° C. and the outlet temperature from this catalyst stage corresponds to the maximum operating temperature of the reactor.

In an embodiment, the pressure at the inlet of the reactor is 0.8-5.0 bar(a), such as 1.5-3.0 bar(a).

In an embodiment, the recycle gas is obtained inside the purification of the hydrocarbons produced and contains an amount of 10-70 wt-%, such as 20-40 wt-%, of C₂ and/or C₄-C₈ olefins.

In an embodiment, the washing agent loaded with methanol, which is produced in a processing unit, is processed in a distillation unit, and the top product of this distillation unit is at least partly directly guided to the reactor as recycle gas, such as to at least one catalyst stage downstream of the first catalyst stage.

In an embodiment, the invention provides a plant for producing olefins from dimethyl ether, such as for carrying out a process described herein, with a reactor which includes at least two series-connected catalyst stages for the conversion of dimethyl ether to olefins, wherein into at least one catalyst stage at least one supply conduit opens for admixing dimethyl ether, recycle gas and/or steam, that into at least one downstream catalyst stage connected with the first stage a conduit opens for feeding in recycle gas, that the recycle gas each contains olefinic, paraffinic and aromatic hydrocarbons, and that at least one control or regulating device is provided for dosing the recycle gas in relation to the dimethyl ether used.

In an embodiment, the control device is designed such that the temperature of the entry stream into the respective catalyst stage and/or of the exit stream of the respective catalyst stage is measured and used as control quantity.

In an embodiment, the supply conduits for the recycle gas are connected with a purification means for processing the olefins or with a distillation unit for the regeneration of methanol washing agent.

### BRIEF DESCRIPTION OF THE FIGURES

Further features, advantages and possible applications of the invention can also be taken from the following description of an exemplary embodiment and the drawing. All features described and/or illustrated form the subject-matter of the invention per se or in any combination, independent of their inclusion in the claims or their back-references.

In the drawing:

FIG. 1 shows the basic process diagram without olefin reactor;

FIG. 2 schematically shows a design of an olefin reactor according to the invention, which adjoins the plant according to FIG. 1.

### DETAILED DESCRIPTION

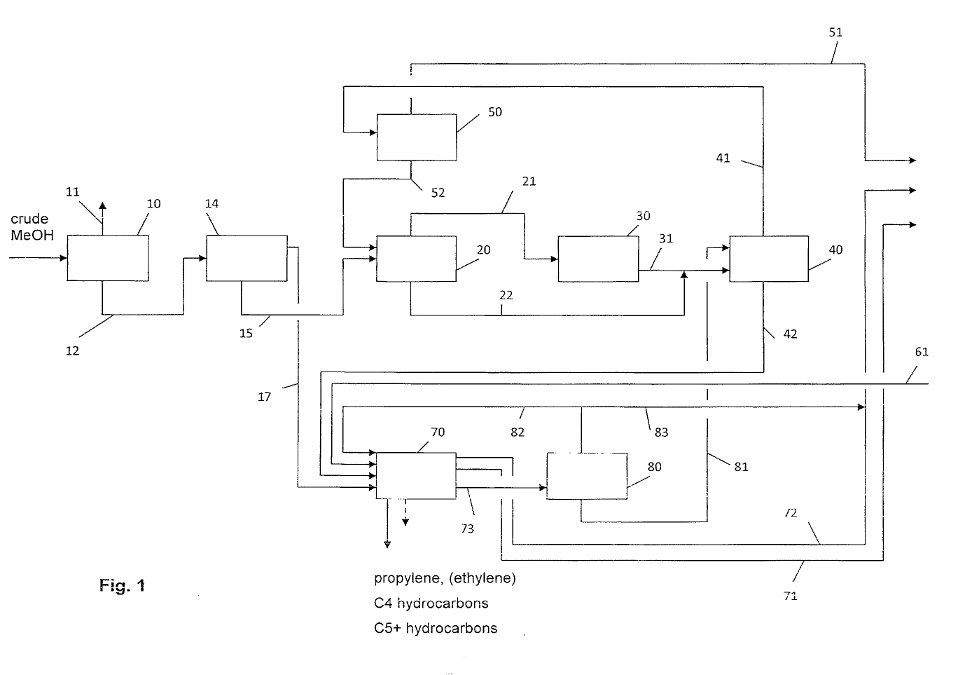

In the plant for producing olefins from crude methanol, which is shown in FIG. 1 in the form of a flow diagram, the crude methanol is fed into a first distillation or rectification column 10. In the usual way known to the skilled person, the column includes a column body, an evaporator in the bottom of the column and a condenser in the top of the column as well as additional internal fittings such as trays, fillings and/or packings. Furthermore, the columns typically are equipped such that only a small part of the top condensate is withdrawn and collected, while the most part again is fed into the top o f the column as reflux, in order to improve the separation effect.

From the rectification 10, gases dissolved in crude methanol and low boilers, in particular short-chain alkanes and oxygenates, can be withdrawn via conduit 11. Via conduit 12, the methanol withdrawn as bottom product of column 10 is fed into a second distillation or rectification column 14. From the bottom of this column 14, a methanol/water mixture is withdrawn via conduit 15 and supplied to the separating device 20. Via conduit 52 the bottom product of column 50, which consists of methanol and traces of DME and water, likewise is charged to the column 20 as reflux. The top product of column 20 chiefly contains methanol, which via conduit 21 is fed into the DME reactor 30 in gaseous form. The water enriched in the bottom is passed on to the column 40 via conduit 22.

In the distillation column 14, pure methanol is obtained via a side draw opening into conduit 17. Pure methanol thus can be exported as by-product. It can, however, also be used as washing agent in a methanol washing column, as is known from DE 10 2011 014 892. Such a non-illustrated column can be integrated into the purification means 70 and be used for removing DME and further oxygenates from crude propylene. In case neither pure methanol is to be exported nor a washing column is to be included in the processing unit 70, the column 14 can be omitted completely. In this case, the bottom product of the column 10 is guided via conduit 12 into conduit 15 and from there directly to the column 20.

Via conduit 31, the dimethyl ether produced in the reactor 30 from methanol by means of heterogeneous catalysis is supplied to a distillative purification in column 40. In column 40, the water which both is contained already in the educt stream as impurity of the crude methanol and is formed during the formation of dimethyl ether is separated from the mixture of methanol and dimethyl ether.

Via conduit 42, the bottom product of column 40, which chiefly consists of water, is supplied to the purification means 70 which also contains a non-illustrated water stripper. This bottom product still contains between 0.5 and 2 wt-% of methanol, which are recovered in the stripper. In principle, there might also be produced a bottom product which still contains only traces of methanol and might directly be supplied to a biological purification. Since the purification means 70, however, already contains a waste water stripper, the illustrated solution is more efficient.

The top product of the column 40 substantially represents a mixture of methanol and DME, which is supplied to a column 50 via conduit 41. The column 50 advantageously is connected with the separating means 20 such that the methanol obtained in the bottom of the separating means 20 is supplied to the separating means 20 as reflux and via conduit 21 thus can likewise be supplied to the conversion to dimethyl ether in the reactor 30. Over head, purified dimethyl ether is discharged from the column 50 via conduit 51, which is obtained at about 10 bar and a temperature of about 50° C. It can thus directly be supplied to the reactor 60 for the conversion of DME to olefins. The purity of the dimethyl ether is 95 to 100 wt-% and is adjustable, in particular with regard to the methanol content, via the reflux ratio of column 50. It should be noted that a minimization of the methanol content (<0.5 wt-%) requires a disproportionately rising reflux ratio, whereby the steam consumption of the column would greatly rise. An amount of 0.1 to 7 wt-%, such as 0.5 to 1.5 wt-% represents an expedient compromise between steam consumption and methanol “slip”. The dimethyl ether furthermore contains small amounts, namely 0.5-2 wt-% of C₄ to C₅ hydrocarbons and further oxygenates as well as traces of water, which substantially have come into the DME synthesis cycle via the methanol. These admixtures do not impair the quality of the dimethyl ether, because according to the invention they are anyway added upstream of the reactor 60 via the recycle gas.

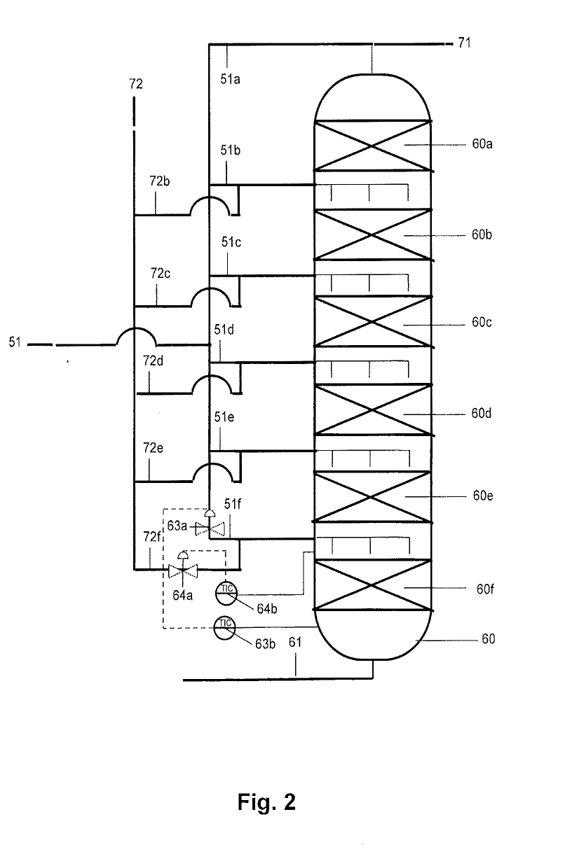

The reactor 60 for the conversion of DME to olefins, which is shown in FIG. 2, is designed as fixed-bed reactor with a plurality of catalyst stages 60a-60f. It is recommendable to use at least 4, more advantageously as shown in FIG. 2, 6 catalyst stages. Charging with dimethyl ether is effected by splitting up the feed stream 51 into the individual streams 51a-51f. At the same time, recycle gas is guided to the reactor 60 via conduits 71 and 72 and in the reactor dosed into the first catalyst stage 60a via conduit 71 and into the catalyst stages 60b-60f via conduits 72b-72f. The recycle gas is produced in the purification means 70 and contains olefinic, paraffinic and aromatic hydrocarbons, such as with C₂ and C₄ to C₈ carbon fractions. The gas also contains small amounts of methanol, which get into the recycle gas via the processing of the recycle gas and the regeneration of the washing methanol.

The recycle gas guided to the first catalyst stage of the reactor 60 via conduit 71 is preheated to the inlet temperature in an undergrate firing unit associated to the processing unit 70. Via conduit 51, a proportional amount of dimethyl ether preheated to 250-350° C. is admixed via conduit 51, so that the mixing temperature of the combined gas stream entering into the reactor 60 is e.g. 474° C.

The supply of dimethyl ether via conduits 51b, 51c and the following as well as the supply of the recycle gas via conduits 72b, 72c and the following to the catalyst stages 60a and the following of the reactor 60 can be effected in different ways. It would be conceivable to charge dimethyl ether and recycle gas to the respective catalyst stage through two separate supply conduits and distributors. A particularly simple design provides to premix DME and recycle gas and charge the same to the catalyst stage via a common distributor. One of the two streams, such as the recycle gas, can be preheated and thus a fine control of the temperature profiles in the catalyst beds can be achieved. Via a non-illustrated gas distributor of the usual construction, the feed gas is distributed over the cross-section of the reactor 60. The individual catalyst stages are connected in series, which is indicated by the connections 62a, 62b and 62c. Due to the mixing of the cold feed gas with the hot reaction gas exiting from the preceding catalyst stage, the latter is cooled and thus in the following reaction stage can react with the admixed dimethyl ether in the desired temperature range.

In principle, it is possible to charge recycle gas with different gas compositions to the individual catalyst stages, wherein as a simple possibility of actuation it is recommendable in particular to charge recycle gas with a different composition to the first catalyst stage, while all succeeding catalyst stages are charged with recycle gas of the same composition. With such a design it is expedient in particular to already charge the entire steam to the first catalyst stage, i.e. via conduit 71 directly to the reactor 60, and to charge a fraction with a minimum methanol constituent to the catalyst stages 2 to n, since thus the high exothermicity as a result of the conversion of methanol can be avoided. In the first catalyst stage, the exothermicity on the other hand can contribute to the “kick-off” of the reaction or allow a rather low inlet temperature, so that charging a stream relatively rich in methanol might be recommendable.

In accordance with the invention it also is favorable to use recycle gas with a particularly high olefin content, in particular with chain lengths C₄ to C₈. Whether C₂ hydrocarbons in the recycle gas also are recirculated to the reactor 60 depends on whether ethylene should be obtained in the plant as by-product or the propylene yield should be maximized. Since these olefins react in the reactor 60 in an endothermal reaction to form propylene (ethylene), they also serve for cooling the system and increase the yield of target product.

The composition of the recycle gas is adjusted in the processing means 70. A configuration of this processing means 70 advantageous for obtaining olefins is described in DE 10 2011 014 892 A1. In principle, the desired main product is separated from the by-products by distillation in the purification means 70. As by-products, liquefied gas and gasoline fractions can be discharged. As recycle streams, hydrocarbon streams and process water streams are provided. Excess process water is discharged. In an embodiment, the product stream is processed in the non-illustrated purification means 70 or according to DE 10 2011 014 892 A1 processed such that a propylene stream with a purity >99.5 wt-%, a fraction of C₄ hydrocarbons and a fraction of C₅₊ hydrocarbons can be withdrawn. From the purification means 70 recycle gas also can be obtained and be guided into the reactor 60 via conduit 71 and conduit 72, wherein it is also conceivable in principle to guide only one recycle gas conduit from the device or to use recycle gas with more than two different compositions, whereby correspondingly more supply conduits would be required.

Finally, the loaded methanol from the methanol washing column is withdrawn from the purification means 70 via conduit 73 and supplied to a distillation apparatus 80. In the top of this column C₄/C₅ hydrocarbons, dimethyl ether and further oxygenates are enriched. This stream advantageously can at least partly be guided directly via conduit 83 as recycle gas to the reactor 60 or via conduit 82 be recirculated into the purification means 70 for further treatment. From the bottom of the column 80 the regenerated methanol is withdrawn via conduit 81. The same contains residual amounts of C₄ and C₅ hydrocarbons as well as water and advantageously is charged into the column 40 as reflux, whereby water is removed from the system, the C₄/C₅ hydrocarbons with dimethyl ether are guided to the reactor 60, and methanol is fed into the DME reactor 30.

Instead of the column 80 for regeneration of the washing methanol it is also possible to use a liquid-liquid extractor according to the prior art. Thereby, an aqueous methanol solution with a relatively large water content is produced. The same can likewise be guided to the column 40 and be processed with a correspondingly higher steam consumption.

In principle it is possible to use several reactors both for the conversion of methanol to DME and for the conversion of DME to olefins.

FIG. 2 once again shows the design of a reactor 60 according to the invention with 6 catalyst stages. Via conduit 51 dimethyl ether is supplied, which via the partial conduits 51a-f is distributed over the individual catalyst stages.

The admixture of recycle gas and steam for the first catalyst stage is effected via conduit 71, while for stages 2 to 6 recycle gas is supplied via conduit 72. The recycle gas for example has a temperature of 98° C., wherein a temperature in the range from 40 to 150° C. is suitable in principle.

The mixture of dimethyl ether and recycle gas is shown in the last stage 60f by way of example. Via a temperature controller 63b and the associated control valve 63a, the DME quantity is adjusted such that after the catalyst bed 60f the desired setpoint temperature of the exit stream is reached. By the cold DME gas supplied via conduit 51f a certain cooling is achieved already, when this stream mixes with the product stream from the upstream catalyst stage 60e, but this cooling is not sufficient. Therefore, recycle gas additionally is dosed in via the valve 64a, so that by means of the temperature controller 64b the desired inlet temperature of the reaction mixture into the catalyst bed also can be adjusted.

This temperature and reaction control concept favorably is carried out in the same way in all other stages, but at least in the stages 2 to 6. The inlet and outlet temperatures of the respective stage are flexible and easily adjustable via the quantity ratio of the respective DME and recycling streams. Thus, an optimum temperature profile for a maximum propylene yield can be adjusted over the entire reactor.

### EXAMPLE

In detail, a reactor as shown in FIG. 2 can optimally be operated with the following settings:

Via the catalyst stages 60a and 60b of the reactor a rising temperature level can be set, and in the second catalyst stage the additional cooling with recycle gas can be minimized. As compared to the following catalyst stages a slightly lower propylene yield then must be reckoned with. The temperature in the catalyst stages 60a and 60b lies between 470 and 500° C.

In catalyst stages 60c to 60e the main conversion of the process takes place, which is distinctly higher than in a conventional execution of the process. As a result of an adjustment of corresponding quantities of cooling gas, the temperature in the catalyst stages 60c to 60e lies between 470 and 500° C.

In the last catalyst stage 60f, a reduced conversion of dimethyl ether or a rather flat temperature interval over the catalyst bed is set. According to the invention, the temperature profile in the catalyst stage 60f for example lies in the range between 480 and 500° C. Thus, at a maximum temperature and with little new formation from DME a rather complete reaction of the C₂ and C₄ to C₈ olefins present in the reaction gas to propylene takes place.

With a conventional design of the reactor, comparable settings only are possible to a limited extent, since in the presence of methanol the exothermicity of the corresponding reaction requires lower inlet temperatures and hence involves a correspondingly reduced propylene yield.

Under otherwise comparable conditions, a propylene yield larger by 3 to 4 wt-% (based on the methanol used) can be reckoned with in the case of a DME-based MTP plant.

The yield improvement is achieved by a combination of the following measures and effects:

The lower heat tonality of the reaction in the case of a conversion of dimethyl ether instead of methanol leads to an increase of the inlet temperatures by 2 to 8° C. and in general to flatter heat profiles shifted towards higher temperatures inside the individual catalyst stages. The reactor thus can be operated at a maximum reaction temperature (i.e. roughly 500° C. on exit from the respective catalyst stage), without the catalyst being damaged.

What is furthermore favorable for the reaction of dimethyl ether to propylene is the increased steam content inside the individual catalyst stages. In the standard MTP process steam is formed by the reaction of methanol. However, this also means that it is only formed inside the respective catalyst stage and the amount thus increases over the respective catalyst stage. In the process according to the invention, however, steam as part of the recycle gas is admixed upstream of the first catalyst stage, which is why this steam is present already before commencement of a chemical reaction.

Furthermore, the use of dimethyl ether allows an optimization of the conversions inside the catalyst stages, without the inlet temperatures for the respective stages having to be lowered very much. Thus, inside the reaction stages a temperature profile is possible, at which the conversion to propylene proceeds with a high selectivity. Furthermore, the use of the last catalyst stage as “post-reaction” stage has an advantageous effect on the total yield, because the amount of C₄₊ olefins in the product streams thus is minimized, as here higher-chain carbon fractions still can be converted to propylene.

The possibility of carrying out the reaction at higher temperatures in addition provides for a lower residence time. At the indicated temperature values, the residence time for example is decreased by 10%. In addition, a lower residence time inside the individual reaction stages now also is possible due to the fact that because of the good control possibilities of the conditions in each individual catalyst stage the conversion can easily be distributed to stages with a less aged catalyst and thus the reserves of catalyst based on the entire reactor can be utilized without breakthrough of major amounts of dimethyl ether and methanol.

### LIST OF REFERENCE NUMERALS

10 rectification column

11-13 conduits

14 rectification column

15-17 conduits

20 separating means

21, 22 conduits

30 reactor

31 conduit

40 rectification column

41 conduit

50 rectification column

51, 52 conduits

60 reactor

60a-60f catalyst stages

61 conduit

62a-62c connection between catalyst stages

63a, 64a temperature controller

63a, 63b control valve

70 purification means

71-73 conduits

80 rectification column

81, 82, 83 conduits

1. A process for producing olefins from dimethyl ether, wherein gaseous dimethyl ether in a purity of 70-100 wt-%, together with recycle gas which contains olefinic, paraffinic and/or aromatic hydrocarbons, as well as steam is charged to a first catalyst stage of a reactor and wherein gaseous dimethyl ether in a purity of 70-100 wt-%, together with recycle gas which contains olefinic, paraffinic and aromatic hydrocarbons is charged to at least one downstream catalyst stage, wherein this downstream catalyst stage additionally is fed with product gas from the upstream catalyst stage.

2. The process according to claim 1, wherein the ratio of the gas streams charged to the first catalyst stage is adjusted such that the amount of dimethyl ether in relation to the recycle gas on entry into the catalyst stage is about 2 to 20 wt-%, and the amount of steam in the entire stream on entry into the catalyst stage maximally is 60 wt-%, and/or that the ratio of the gas streams charged to a downstream catalyst stage is adjusted such that the amount of the recycle gas in relation to the dimethyl ether on entry into the catalyst stage is 10-200 wt-%.

3. The process according to claim 1, wherein the ratio of dimethyl ether and recycle gas is adjusted such that the inlet temperature into the catalyst stage lies between 440 and 490° C. and/or the outlet temperature from the catalyst stage lies between 460 and 520° C.

4. The process according to claim 1, wherein the ratio of dimethyl ether and recycle gas is adjusted such that the temperature over the catalyst stage rises by 5 to 100° C.

5. The process according to claim 1 wherein recycle gas with a different composition is charged to the first catalyst stage, while all succeeding catalyst stages are charged with recycle gas of the same composition.

6. The process according to claim 1, wherein a mixture of dimethyl ether and recycle gas is charged to at least two catalyst stages, wherein the respectively added recycle gas has a different composition.

7. The process according to claim 1, wherein for the last catalyst stage the ratio of dimethyl ether and recycle gas is adjusted such that the temperature over the catalyst stage rises by 5 to 20° C. and the outlet temperature from this catalyst stage corresponds to the maximum operating temperature of the reactor.

8. The process according to claim 1, wherein the pressure at the inlet of the reactor is 0.8-5.0 bar(a).

9. The process according to claim 1, wherein the recycle gas is obtained inside the purification of the hydrocarbons produced and contains an amount of 10-70 wt-%, of C₂ and/or C₄-C₈ olefins.

10. The process according to claim 1, wherein the washing agent loaded with methanol, which is produced in a processing unit, is processed in a distillation unit, and the top product of this distillation unit is at least partly directly guided to the reactor as recycle gas.

11. A plant for producing olefins from dimethyl ether, for carrying out a process according to claim 1, with a reactor which includes at least two series-connected catalyst stages for the conversion of dimethyl ether to olefins, wherein into at least one catalyst stage at least one supply conduit opens for admixing dimethyl ether, recycle gas and/or steam, that into at least one downstream catalyst stage connected with the first stage, a conduit opens for feeding in recycle gas, that the recycle gas each contains olefinic, paraffinic and aromatic hydrocarbons, and that at least one control or regulating device is provided for dosing the recycle gas in relation to the dimethyl ether used.

12. The plant according to claim 11, wherein the control device is designed such that the temperature of the entry stream into the respective catalyst stage and/or of the exit stream of the respective catalyst stage is measured and used as control quantity.

13. The plant according to claim 10, wherein the supply conduits for the recycle gas are connected with a purification means for processing the olefins or with a distillation unit for the regeneration of methanol washing agent.

14. The process according to claim 2, wherein the ratio of the gas streams charged to the first catalyst stage is adjusted such that the amount of dimethyl ether in relation to the recycle gas on entry into the catalyst stage is about 5-15 wt-%.

15. The process according to claim 2, wherein the amount of steam in the entire stream on entry into the catalyst stage maximally is 50 wt-%.

16. The process according to claim 2, wherein the ratio of the gas streams charged to a downstream catalyst stage is adjusted such that the amount of the recycle gas in relation to the dimethyl ether on entry into the catalyst stage is 15-50 wt-%.

17. The process according to claim 4, wherein the temperature over the catalyst stage rises by 5 to 40° C.

18. The process according to claim 4, wherein the temperature over the catalyst stage rises by 10-25° C.

19. The process according to claim 8, wherein the pressure at the inlet of the reactor is 1.5-3.0 bar(a).

20. The process according to claim 1, wherein the washing agent loaded with methanol, which is produced in a processing unit, is processed in a distillation unit, and the top product of this distillation unit is at least partly directly guided to the reactor as recycle gas to at least one catalyst stage downstream of the first catalyst stage.
